(19) United States
(12) Patent Application Publication
(10) Pub. No.: US 2011/0243993 A1
(43) Pub. Date: Oct. 6, 2011

(54) Method for diagnosing and creating immunogenic tolerance in contact allergy and other epithelial immunotoxicological ailments

(76) Inventors: Kerstin S. Broo, Lindome (SE); Sofia I. Andersson, Goteborg (SE); Anna-Lena Stenfeldt, Goteborg (SE); Charlotte A. Jonsson, Goteborg (SE)

(21) Appl. No.: 13/065,690

(22) Filed: Mar. 28, 2011

Related U.S. Application Data
(60) Provisional application No. 61/341,359, filed on Mar. 29, 2010.

(51) Int. Cl. A61K 39/35; C40B 30/04; C40B 40/10

(52) U.S. Cl. 424/275.1; 506/9; 506/18

(57) ABSTRACT

A method is provided for diagnosis and induction of immunogenic tolerance in contact allergy and other epithelial immunotoxicological ailments, based on knowledge of the role that keratinocytes play in these ailments and is of importance for several conditions, including but not limited to allergic contact dermatitis (ACD), drug hypersensitivity reactions (DHRs) and autoimmune diseases.

### CROSS-REFERENCE TO RELATED APPLICATIONS

This application claims priority from U.S. Provisional Application Ser. No. 61/341,359 filed Mar. 29, 2010.

### FIELD OF THE INVENTION

The present invention relates generally to immunology. More specifically the invention relates to allergic contact dermatitis (ACD) and new ways to diagnose and create immunogenic tolerance in ACD and other epithelial, immunotoxicological ailments.

### BACKGROUND OF THE INVENTION

The skin is the principal epithelial barrier between self- and non-self and it protects us from the harmful effects of exposure to e.g. microorganisms, chemicals, and UV radiation. Sometimes the skin fails to protect against the compounds around us and that can result in, among other things, irritant contact dermatitis (ICD) or allergic contact dermatitis (ACD). ACD is a permanent, specific immunologic hypersensitivity reaction, whereas ICD is a non-immunological local inflammatory reaction. ACD affects 15-20% of the population in the Western world and it is the most prevalent form of immunotoxicity found in humans. As ACD is a common occupational injury, there are substantial psychosocial and socioeconomic effects. ACD is caused by small molecules (haptens) that breach the skin barrier. Once an individual is sensitized, there is today no cure, but the offending compound and any cross-reacting compounds will have to be avoided. This can be very difficult to do for ubiquitous allergens such as nickel, preservatives and fragrances. The growing list of compounds that can cause ACD currently comprises >4000 substances. In order to predict the sensitizing properties of a compound, the OECD approved benchmark test today is the murine Local Lymph Node Assay (LLNA).

Our invention makes it possible to cure/prevent ACD. Today, there is no cure for ACD, and since 15-20% of the population is affected, there are major socioeconomic and psychosocial problems. Furthermore, our invention makes it possible to diagnose which compounds a patient is allergic to through a simple blood test. This could replace patch testing as the diagnostic method. In vivo testing (including, but not limited to, patch testing) has many drawbacks, a crucial one being that the doctor runs the risk of sensitizing the patient (i.e. causing allergy). Since our findings also show that in vivo diagnostic procedures could instigate autoimmune, autotoxic diseases, and/or chronic inflammation, it is no longer ethical to continue in vivo testing. There is thus an urgent need for in vitro tests, something that the invention herein provides.

The skin is a stratified tissue, and the outermost layers are called the epidermis. The epidermis, in turn, can be subdivided into a dead layer (the stratum corneum, SC) and the living epidermis. The epidermis is mainly (˜95%) composed of keratinocytes. These are interspersed with the specialized antigen-presenting cells (Langerhans cells), Merkel cells and melanocytes. Keratinocytes get their name from their large amount of type I and II intermediate filaments called keratins. These structural proteins are essentially what give the epidermis (and epithelia) high tensile strength. Epithelial tissues express different pairs of keratins depending on the cell type and the stage of differentiation. The keratinocytes in the basal layer of stratified epithelia express chiefly the keratin 5/keratin 14 (K5/K14) pair and up to 35% of the protein content is K5 and K14. This pair is replaced by the K1/K10 pair in the suprabasal layers, which accounts for as much as ˜80-85% of the total cellular content of a fully differentiated keratinocyte. In summary, the epidermis is mainly composed of keratinocytes that in turn have a very high content of keratins.

Keratins form bundles of insoluble filaments to afford an intracellular skeleton. When cells in the basal layer (the only mitotic layer in the epidermis) go through apoptosis (as a part of normal growth regulation), there is a mechanism in place to deal with the insoluble keratins that would otherwise form amyloid plaques. Basal keratinocytes that go through apoptosis thus dispel keratins, along with other cellular remnants, in apoptotic blebs. In skin, these are called keratin bodies. Keratin bodies drop through the basement membrane and are then phagocytosed by dendritic cells in the dermis. This process is facilitated by opsonization.

ACD has been shown to be mediated by T-cells. T-cell mediation comes about through MHC (major histocompatibility complex) restriction and presentation of peptides to naïve T-cells in the lymph nodes. Since MHC molecules present peptides on the order of 8-10 amino acids (MHC class I), or 9-25 amino acids (MHC class II), a hapten is thus too small to warrant a T-cell mediated immune response in itself. Instead, in a seminal paper published as early as 1935 by Landsteiner and Jacobs, the key step in sensitization is proposed to be the formation of the complete allergen through covalent linkage of the hapten to a carrier protein turning self into non-self. Inspection of the list of known haptens reveals common traits: haptens are typically small (≦1000 Da), hydrophobic (log P≧2), and they frequently contain an electrophilic group. The covalent linkage that turns self into non-self is therefore primarily envisioned as a reaction between an electrophilic group on the hapten and a nucleophilic moiety in a protein. Alternatively, in the case of haptens containing not one (monodentate) but two reactive groups (bidentate), a cross-linking of two self-proteins could also occur. Seemingly innocent compounds that do not contain electrophilic moieties can also be allergenic. It has been proposed that these compounds can be autoxidized or photo-oxidized outside the body (prehaptens) or metabolized in the skin (prohaptens) to form potent haptens.

The currently accepted theory is that a compound causes ACD through the following steps:
Penetration of the compound through the stratum corneum into the viable epidermis, followed by covalent conjugation to proteins. Uptake/processing of haptenated proteins by skin-residing antigen presenting cells. The focus has been on the Langerhans cells (LCs) of the epidermis, but dermal dendritic cells (DCs) have been gaining more attention lately. Subsequent hapten-induced maturation and migration of LCs. Mature LCs present the MHC class I or II restricted (depending on the nature of the hapten) haptenated peptide to naïve T cells in the draining lymph nodes. Helper T cells (T_{H}) and/or cytotoxic T cells (T_{C}) proliferate and enter the circulation.
The next time the hapten is encountered; the allergy is elicited. The only way to avoid the allergic symptoms is henceforth to avoid the hapten and any cross-reacting compounds.

As stated above, the key step in sensitization is proposed to be the formation of a complete allergen through the covalent linkage of the hapten to a carrier protein thus turning self into a non-self. To decode sensitization, haptenated proteins need to be singled out with molecular precision against the entire backdrop of the proteome ending up with identification of the targeted protein(s) and the exact position of haptenation. This has not been previously achieved.

DHRs (drug-induced hypersensitivity reactions) are a major problem and the mechanisms behind DHRs are not known to a full extent. DHRs represent approximately ⅓ of all adverse drug reactions, are sometimes life threatening, can require hospitalization, and entail changes in drug prescription. This is an important public health problem, since more than 7% of the population is concerned. DHRs are unfortunately unpredictable and prospective clinical studies are very difficult to perform. The use of animal models (rats, guinea pigs, cats, dogs) is almost the only way to test the likelihood of a compound to cause DHRs. However, since DHRs are also unpredictable in animals, it is difficult to obtain and know beforehand if a certain animal model will correctly predict the effect in humans.

The findings presented herein that keratins (and other proteins released in blebs) are key proteins involved in ACD are utilized in diagnostic kits for in vitro testing. These tests can identify and diagnose the compounds, and/or the cryptic epitopes, and/or the neoepitopes to which a patient is allergic. The tests will replace the so-called “patch testing” that takes place in the clinic today. Replacing patch testing is an urgent need since it has been shown in the present invention that autoimmune diseases can be instigated through epithelial contact with haptens, i.e. the compounds used in the patch tests. The findings presented herein that keratins (and other proteins released in blebs) are key proteins involved in ACD is utilized, through immunogenic tolerance principles, to minimize or alleviate ACD. This has not been possible before, as it is not ethical to administer the reactive compounds (the haptens) themselves, but a “dead” compound that is already coupled to its primary target(s) or mimics thereof is much more harmless. The invention represents substantial advancements since neither in vitro diagnosis nor personalized medicine with regard to ACD has been possible before as the biological processes have not been previously known.

The invention may be used in diagnosing allergic contact dermatitis. Today, patch testing is the way to diagnose the compounds to which a patient is allergic. This test is performed in the following way: On the first visit to the doctor, substantial amounts of known haptens are applied to the upper back of a patient. The compounds are kept under occlusion and left undisturbed for at least 48 hours. This is to ensure a good uptake. At the second appointment (two days later) the compounds are removed. To identify the test sites, the back is marked with a suitable marker such as an indelible black felt tip pen. At the third appointment (usually 4 days after application), the back is checked again. A record of the back is made by the dermatologist (medical doctor) usually at both 48 hours and 96 hours post-application. Signs that are recorded include sweat rash, follicular pustules, burn-like reactions, pink areas, elevated pink or red plaques, ‘papulovesicles’ blisters or ulcers. The responses are then recorded are labeled as: Negative (−), Irritant reaction (IR), Equivocal/uncertain (+/−), Weak positive (+), Strong positive (++), Extreme reaction (+++).

There are several drawbacks to patch testing (and in vivo testing procedures):
The risk of sensitizing an individual. This means that the medical doctor runs the risk of instigating contact allergy to new substances that the patient was not allergic to before the patch test. After all, known haptens that are commonly encountered (that is why they are on the standard lists) are actually put in direct contact with the skin under optimal conditions (occlusion for days when the patient is not allowed to shower) to allow for an optimal response.
The risk of instigating other ailments such as autoimmune diseases and also priming the patient for DHRs. Our findings show that the same epitopes are released again, and again upon epithelial contact, and that this also represents a breach of self-tolerance as autoantibodies are produced. Thus, by subjecting the patient to a patch testing procedure, where a large number of known haptens is applied at the same time and really given time to penetrate through the epithelial barrier under optimal conditions, there is a considerable risk that the sheer amount of released self-proteins will lead to crossing over the so-called immunological threshold. Once that happens, self-tolerance is breached and autoimmune diseases (and possibly DHRs) can follow. Furthermore, the risk of instigated systemic amyloidosis/autotoxic diseases through in vivo testing cannot be ruled out. The basis for this is that some sensitizing molecules instigate an increased serum level of the acute-phase protein SAP and of antinuclear antibodies (ANA). Normally, the plasma concentration of SAP increases in response to inflammation and it binds all types of fibrils, including keratin intermediate filaments (KIFs) and elevated levels of ANA is a marker for autoimmune diseases.
Patch testing is a procedure that is highly cumbersome, sometimes painful, and definitively time-consuming for a patient. It also involves having potentially harmful compounds on the back for days without being allowed to shower that portion of the body. The patient also has to return to the clinic several times. There is a high probability that the allergenic compound is not picked up in an initial screen, and that the whole procedure needs to be repeated with other haptens, even several times. In fact, the standard series in Europe consists of only the 24 most common haptens, whereas the list of known haptens is on the order of 4000 molecules. It can also involve that the patient has to bring in his/her own products both household products and/or work products. Despite these efforts, it may in the end, with conventional methods, turn out to be impossible to actually diagnose which allergens to which the patient responds.
Furthermore, patch testing is a very cost-inefficient method, as it involves numerous specialist visits, and a lot of hands on time, not only from medical doctors and nurses and administrative personnel, but also from other specialists that can inspect workplaces and the like, trying to identify the compound responsible for the allergic symptoms.

An additional uncertainty is that the interpretation of the results requires considerable experience and training, and the results are in that way not objective but instead subjective. This might mean that the wrong diagnosis is made, and the patient (and his/her employer, etc.) might proceed with insufficient and/or unnecessary changes in the home and workplace environment. A further problem is that some haptens deteriorate and/or react with something else, either during their shelf-life in the formulation, and/or in the ointments applied on the patient during the prolonged exposure in the patch test. Thus it is not always known exactly what is applied and at what concentrations.

Thus, an in vitro diagnostic test is highly desirable. The only in vitro diagnostic test today is a test for nickel allergy. Our findings make it possible to prepare in vitro diagnostic kits that circumvent these obstacles. The patient does not have to have any compounds on his/her back, but he/she only has to submit a blood sample and the diagnostic step takes place outside the body. In addition to removing the risks and substantially lowering the time and nuisances a patient has to go through, there is a substantial economical benefit as it reduces the input from specialists and medical personnel. In addition, as the screening for allergens can be done a high throughput format, there is also the benefit of allowing the screening of a vast number of haptens, and self-epitopes neo/cryptic epitopes and thus the medical doctor has a much more complete view of the health status and future risks of that particular person. Furthermore, as it is an objective test, the patient and his/her employer have better information for making the correct changes in his/her environment.

In summary, it has not previously been possible to diagnose the ACD status in vitro. However, solid evidence of the mechanisms in place during sensitization is presented, and an in vitro test concerning the health status in ACD and other ailments caused by epithelial contact with various compounds has been invented. The invention uses keratins and other proteins that have been identified as being released in the blebs from keratinocytes as the basis of the test. Epitope mapping (antibodies and/or T-cells) using blood/serum samples from patients is used in the invention. The proteins (including keratins and other proteins and/or peptides with sequences based on these proteins) are treated in different ways including: untreated, covalently modified with known haptens, and cross-linked, not excluding other formats). The response when serum (blood) samples from patients are added to these protein/peptide arrays is measured. If the patient has antibodies/T-cells towards a target, then the test point toward that target is a culprit in the medical condition(s).

There is no cure for ACD today. Avoiding the compound in question, and any cross-reactive compounds, is the only option. The findings presented herein fortunately also open up a solution in treatment of ACD, using immunological tolerance principles based on the information acquired from the diagnosis using the invention.

### SUMMARY OF THE INVENTION

An object of the present invention is to use the knowledge of keratinocytes' role in allergic contact dermatitis (ACD) to create new ways of diagnosing the allergens in ACD.

Another object is to develop in vitro diagnostic kits that can be used to estimate the extent to which a patient has breached his/her self-tolerance, i.e. the risk of that patient developing autoimmune diseases.

Another object is to develop in vitro diagnostic kits that can be used to estimate the risk of that patient showing DHRs.

Another object is to use the invention to diagnose the allergens in allergies contracted through contact between a compound and epithelial cells (oral, eyes, through inhalation, etc.).

Another object is to use the invention to screen which compound(s) a patient is allergic to, which neoepitopes a patient is allergic to, and which cryptic epitopes to which a patient is allergic.

Another object is to use the invention to induce tolerance (i.e. “vaccination”) to compound(s), cryptic epitopes and neoepitopes by immunological tolerance principles.

Another object is to treat ACD (and/or other conditions such as autoimmune diseases, DHRs, etc.) through immunogenic tolerance.

Other objects and advantages of the present invention will become obvious to the reader and it is intended that these objects and advantages are within the scope of the present invention.

### BRIEF DESCRIPTION OF THE DRAWINGS

FIG. 1A) illustrates the events in skin upon contact with sensitizers. B) The blebbing response of keratinocytes in vitro.

FIG. 2 illustrates the protein/peptide arrays for in vitro diagnostics. It shows an array coated with amino acid containing epitope targets. A serum sample is run over the array and the antibody profile is detected with a secondary detection antibody conjugated to a fluorescent molecule or an enzyme.

### DETAILED DESCRIPTION OF THE INVENTION AND PREFERRED EMBODIMENTS THEREOF

### Definitions

As used herein the terms “keratinocytes” or “keratinocyte cells” is intended to mean primary human keratinocytes, or keratinocytes from other mammals. The term may further include primary, immortalized and stem cell culture systems such as adult primary human epidermal keratinocytes (HEKa) or neonatal primary human epidermal keratinocytes (HEKn)s or cell lines with HEKn characteristics, such as HaCaT.

As used herein the terms “keratin” or “keratins” is intended to mean the keratin protein family of fibrous structural proteins being one of the key structural components of skin, hair and nails. The term is predominantly intended, but not limited, to mean the specific keratin subtypes keratin 1, 5, 8, 10, 14 and 18.

As used herein the term “hapten” is intended to a xenobiotic compound that can form immunogenic complexes with endogenous macromolecules, e.g. proteins, in the human body and activate the adaptive immune system. Generally the term is referred to a xenobiotic compound able to cause e.g. contact allergy when breaching the skin barrier, reacting with proteins therein, but may also refer to compounds with other entrance routes e.g., but not limited to subcutaneously, intravenously, per orally.

As used herein the term sensitizing compounds is intended to mean compounds that causes a substantial proportion of exposed individuals to develop an allergic reaction in normal tissue after repeated exposure.

As used herein the term “haptenation” is intended to mean the reaction and complex formation between a hapten and a macromolecule, e.g. a protein, and “haptenated protein” is intended to mean the formed hapten-protein complex, i.e. a protein that has bound a hapten.

As used herein the term “haptenation site” or “haptenation location” is intended to mean a specific amino acid of the specific protein with which the hapten reacts, i.e. the exact location of the hapten on the protein.

As used herein the terms “blebs” or “blebbing” are intended to mean the formation and release of membrane vesicles from cells. Blebbing is the process where membrane protrusions are formed. Blebs are protrusions formed at plasma membrane of a cell, which eventually dissociate or bud off from the cell into the surrounding medium. Blebs are in general of spherical nature and consist of the plasma membrane bilayer with a diameter of about 1 μm to about 100 μm. The term “keratinocyte blebbing response” is intended to mean the cellular expulsion of membrane lipid vesicles specifically from keratinocytes or keratinocyte-type cells described previously.

As used herein the term “sensitization” is intended to mean the immunological process by which an individual is exposed to e.g. a contact allergenic compound, i.e. a hapten, that by forming immunogenic complexes with macromolecules activates the adaptive immune system.

As used herein the term “elicitation” is intended to mean the cutaneous inflammatory reaction of an individual due to re-exposure of a compound to which the individual is sensitized or due to the exposure of any crossreacting compounds.

As used herein the term “protein derivatization” is intended to mean the event of a compound (e.g. a hapten) binding to a protein.

As used herein the term “allergic contact dermatitis (ACD)” is intended to mean the clinical manifestation of the cutaneous immunological hypersensitive reaction that an individual develops when re-exposed to a contact allergen (hapten) to which the individual is sensitized, or any crossreacting compounds.

As used herein the term “drug hypersensitivity reaction (DHRs)” or “protein drug-induced hypersensitivity reactions” is intended to mean an immune-mediated reaction to a drug, in which the drug act as hapten, binding covalently endogenous proteins causing sensitization, and at subsequent re-exposure, elicitation.

As used herein the term “autoimmune diseases” is intended to mean a disease in which the immune response reacts against endogenous substances, constituents and tissues normally present in the body. This will include, but is not limited to diseases such as Rheumatoid arthritis, SLE (systemic lupus erythematosus), Sjögrens Syndrome (SS), type I diabetes diabetes and Psoriasis.

As used herein the term “epitope mapping” is intended to mean the process of determining to what protein epitopes antibodies or T-cells are directed against in a sensitized individual.

As used herein the terms “cryptic epitopes and “neoepitopes” are intended to mean haptenated peptides and proteins, and/or proteins and peptides that have not been processed as they normally would have been, and or cross-linked proteins. These epitopes are thus normally concealed from the immune system.

As used herein the term “epitope spreading” is intended to mean the development of immune responses to epitopes distinct from, and noncross-reactive with, the dominant epitope.

As used herein the term “blood-derived sample” is intended to mean any sample derived from blood, including but not limited to, whole blood, serum, plasma, cells derived from blood (e.g. peripheral blood mononuclear cells) and other purified constituents from blood such as antibodies and cells. The invention can also be used for other biological samples that contain e.g. antibodies or T-cells, including but not limited to tissue, liquid tissue and secretions from the body of a subject/patient.

Contrary to common belief, the inventors have surprisingly found that in human ACD, keratinocytes play a crucial role in the sensitization process. This comes about through an unfortunate clash between culture and evolution. The evolutionary defense systems of human skin are aimed at large parasites, viruses and microorganisms. However it can be shown that this results in ideal, stratified physio-chemical properties that allow penetration of reactive haptens down to, and into the basal keratinocytes. Furthermore, it has been found that, in addition to other proteins, important targets are keratins, and specifically keratins 5 and 14 (K5 and K14) in human skin excerpts. In particular, the amino acid cysteine 54 (C54) of K5 has been identified as a target for thiol-reactive haptens in human tissue and primary human keratinocytes. This residue is situated in a stretch of amino acids that makes crucial non-covalent contacts with the C-terminal tail of desmoplakin, thereby anchoring the cell with its surroundings through desmosomes (cell-cell) and hemidesmosomes (cell-basement membrane). An alteration of nearby residues has been shown to substantially reduce the keratin-desmoplakin interaction, thereby reducing the cell-cell and cell-matrix interactions, creating in effect a homeless cell. If an epithelial cell loses its contacts with its surrounding, a growth regulation process is in place to prevent erroneous colonization. Thus, a homeless epithelial cell is doomed to anoikis, i.e. apoptosis brought on by “being without a home”. During apoptosis, ball-shaped, membrane-enclosed so called “blebs” appear on the surface of the dying cell. The cell then breaks down into smaller fragments called apoptotic bodies. Phagocytic cells then swallow up the apoptotic bodies without causing an inflammatory reaction. Blebbing is a common phenomenon; in addition to apoptosis it happens for example during the cytokinesis phase of cell division, in nonmotile embryonic blastomeres, and in some cell migration events.

Through studies on human keratinocytes, it can be shown that haptens covalently modify keratins (and other proteins), and that thiol-reactive haptens, such as mBBr, modify C54 of K5. Furthermore, it has been shown, through studies on human keratinocytes that haptens, directed against different nucleophiles in proteins, bring on blebbing, thus releasing large quantities of previously cryptic epitopes and also neoepitopes (e.g. haptenated peptides and proteins, and/or proteins and peptides that have not been processed as they normally would have been, and or cross-linked proteins). Through Western blot experiments, it is shown that blebs from the predominantly amine-directed hapten oxazolone and the predominantly arginine-reactive hapten glyoxal also contain K5 and K14. Furthermore, it is also shown that irritants (e.g. sodium dodecyl sulphate, SDS) and non-allergenic compounds (e.g. nonanoic acid) do not initiate blebbing. It is shown that the theory is relevant in an in vivo setting by performing ELISA experiments on sera from experimental animals (mice). In sera from animals that had been exposed to sensitizing compounds (via skin contact) antibodies against K14 were detected. Furthermore, it was found that contact with some sensitizing compounds (including, but not limited to, glyoxal, CDNB (1-chloro-2,4,-dinitrobenzene and dBBr (dibromobimane) results in increased levels of acute-phase proteins such as serum amyloid P component (SAP). Normally, the plasma concentration of SAP increases in response to inflammation. It is known that chronic inflammation can lead to self attack (autotoxicity) due to the innate immune defense. An increased level of SAP is a common feature in diseases such as Alzheimer's. We have also shown that contact with some sensitizing compounds (including, but not limited to, dBBr, CDNB, oxazolone and glyoxal) results in increased levels of anti-nuclear antibodies (ANA). Elevated levels of ANA are common features in various autoimmune diseases such as Sjögrens syndrome (SS), rheumatoid arthritis (RA) and systemic lupus erythematosus (SLE).

Based on the literature, covalent modification, in all likelihood, short circuits the defense mechanisms that prevent erroneous colonization by homeless epithelial cells. Anoikis is thus executed. The dying keratinocytes bleb off keratin bodies (or the like), that drop below the basal membrane, where their remnants are devoured by dermal dendritic cells. Crucially, these blebs now contain, among other cellular remnants, haptenated i.e. non-self proteins including keratins as well as other proteins. This process thus results in exposing the immune system to a large amount of cryptic epitopes, and furthermore, some of the proteins are haptenated i.e. non-self. Haptenation could also affect the processing of proteins such that neoepitopes are exposed. For bidentate haptens, self-proteins could also become cross-linked thus also creating neoepitopes. All of these aspects likely contribute to sensitizing the individual. It is not excluded that smaller blebs, including, but not limited to exosomes, might also contribute to sensitization.

In summary, haptens penetrate down to the basal layer, enter the basal keratinocytes, and react with K5 and K14 (and other, less abundant proteins), thus bringing on blebbing, resulting in the formation of keratin bodies (blebs), that drop below the basal membrane, where the neo and/or cryptic epitopes are phagocytosed by antigen-presenting cells (mainly dermal dendritic). This results in sensitization. Elicitation occurs when the haptens again derivatize keratins, keratin-derived peptides and/or other proteins, probably both in the stratum corneum and in the basal layer. Other proteins may of course also be derivatized; however keratinocytes make up ˜95% of the cells in the epidermis, and keratins make up ≧80% of the total cellular protein in a fully differentiated keratinocyte. Of note is that K5 is homologous to other type II keratins such as K1, and K14 is homologous to other type I keratins such as K10. The K1/K10 pair is expressed in the suprabasal layers. Thus, in the elicitation phase, when a compound reacts with K1 and K10 in the suprabasal layers, homologous epitopes are produced. Both in sensitization and elicitation, the body will thus be subjected to large amounts of highly homologous and/or identical and recurring epitopes, as keratins are derivatized.

Furthermore, emerging from the work presented herein is a theory of recurring epitopes upon epithelial exposure to chemical compounds. All stratified, squamous epithelial basal cells express the target proteins in high abundance (up to 35% of the cellular content). Epithelial cells line the cavities and surfaces of structures throughout the body and many glands are also formed from epithelial tissue. For example, K5 antibodies stain cells in tissue such as the: bronchus (respiratory epithelial cells), cervix, epididymis, esophagus, nasopharynx, oral mucosa, prostate, salivary glands, urinary bladder, squamous epithelial cells in the tonsils, vagina and vulva/anal skin. Macrophages in the lung are also stained. Human corneal epithelia also stain for K5. As K14 (and K15) is expressed in a pair with K5, K14 is also present in these tissues and cells. In combination with the fact that simple epithelial cells express the homologous K8/K18 pair, the results thus indicate a general mechanism whereby the identical or highly homologous epitopes are encountered irrespective of the route of entry of the compound into the body. The findings presented herein can therefore also be utilized in diagnostic kits for in vitro diagnosis of various ailments, including for example chemically induced asthma and drug hypersensitivity reactions, not excluding other ailments that can be caused through epithelial contact with a compound. The findings can also be utilized, through immunogenic tolerance principles, to minimize or alleviate the same ailments. Neither of these has been possible before, since the biological processes have not been known before.

In addition, the finding that the identical or highly homologous epitopes are encountered irrespective of the route of entry of the compound into the body (exemplified by K5/K14 and K8/K18) has other implications. Thus, when epithelial contact (such as via the skin) with a compound has caused basal keratinocytes to die and release blebs, autoantibodies (and likely other parts of the immune system including, but not limited to, T-cells) against keratins (and other proteins in the blebs) are produced, and the individual has now been sensitized. If another, unrelated compound such as a drug is ingested/inhaled/or otherwise then comes into contact with epithelial tissue, it can react with basal epithelial cells, and it too can cause blebbing. The same epitopes that the individual already has autoantibodies against are again released in the blebs. The circulating memory T-cells and/or the autoantibodies against the protein epitopes are then recruited and a drug hypersensitivity reaction (DHR) is observed. Thus, for some DHRs (including, but not limited to, fixed-drug eruptions) it is highly likely that the same mechanisms, with regards to how a compound interacts with epithelial cells, are operating. These findings explains why DHRs can be observed even when the individual never has been exposed to the drug in question before, as the response can be largely directed against the released self-proteins. This hypothesis is also corroborated by the finding that basal keratinocytes are targeted in drug-induced hypersensitivity reactions (“Incidence of circulating antibodies reactive with basal cells of skin in drug reactions.” T. van Joost, Acta Dermatovener (Stockholm) 54: 183-188, 1974). Our invention can therefore provide new possibilities for diagnostic kits for in vitro testing of possible DHRs in a relevant system. Our findings can also be utilized, through immunogenic tolerance principles, to minimize or alleviate DHRs. The invention presented herein thus represents major advancements in in vitro tests and treatments for adverse drug reactions.

Furthermore, the present inventors have shown that haptenation results in the release of cryptic epitopes, otherwise non-accessible to the immune system and/or neoepitopes that have been differently processed. This is important not only for contact allergy, but it has dire implications concerning the breach of self-tolerance in autoimmune diseases. This hypothesis is borne out by our findings of autoantibodies against K14 in murine sera. That is, the ELISA measures not antibodies against haptenated proteins, but antibodies towards the proteins themselves. Even though the experiments, for ethical reasons, are performed on mice, it is highly likely that humans will also develop autoantibodies upon skin contact with sensitizers. Many processes can occur in the body once blebbing is instigated and self-tolerance is breached (i.e. breach of tolerance against K5 and K14, breach of tolerance against other proteins found in the blebs (e.g. calmodulin, annexin, cofilin, peptidylprolyl-cis/trans isomerase A, transitional endoplasmic reticulum ATPase), epitope spreading through homology, release of other cellular debris like ribonuclear proteins, etc.). Examples of these autoimmune diseases include SLE (systemic lupus erythematosus); rheumatism (RA); Sjögrens Syndrome (SS); and type I diabetes, but do not exclude other autoimmune diseases. Notably, in these diseases (and other autoimmune diseases) autoantibodies against keratins and other bleb proteins have been reported in the literature. Also, keratinocytes are targets of autoantibodies found in sera from patients with atopic dermatitis. Thus, the present findings of how certain compounds can, through epithelial contact, cause breach of self-tolerance and therefore autoimmune diseases, is the basis also for diagnostic kits and tolerance towards targets in autoimmune diseases. Since this is a new finding and a new theory, there are currently no tests that provide this detailed understanding.

Furthermore, glycine-rich cell wall protein (GRP) is a ubiquitous food protein that has a high homology with keratins and other self proteins (e.g. Epstein-Barr virus nuclear antigen-1 (EBNA-1), heterogeneous nuclear ribonucleoprotein and fibrillar collagen), which are common targets in autoimmune disorders. Antibodies against a peptide epitope derived from GRP and peptide-specific T cell clones can be found in sera from patients with autoimmune disorders (RA, SLE, Psoriatic arthritis PsA, Chronic idiopatic urticaria CIU), CPI (Chronic parvovirus B19 infection) and food allergies (cereals, fish, fruit and vegetables). However, normal subjects do not show these immune responses (“Glycine-rich cell wall proteins act as specific antigen targets in autoimmune and food allergic disorders.” C. Lunardi, et. al., International Immunology, Vol. 12, No. 5, pp 647-657). The anti-GRP peptide antibodies purified from the sera specifically recognized EBNA-1 and autoantigens such as keratin, collagen and actin. Thus, antigen spreading through a common (homologous) sequence among apparently divergent proteins from plants, viruses and humans may be responsible for autoimmune diseases as well as food allergies. Importantly, it is quite conceivable that this constitutes an example of epitope spreading, not from GRP, but from keratins and other proteins released in blebs through the action of sensitizing molecules (haptens) upon epithelial exposure. Interestingly, parvovirus B19 has been associated with a large range of autoimmune diseases, including but not limited to, Kawasaki disease, mixed connective tissue disorder, dermatomyositis, autoimmune thyroid disease, autoimmune schizophrenia, and polyarteritis nodosa. It is possible that antibodies against keratins (and other proteins released in blebs) through cross reactivities with parvovirus B19 play a role also in these cases. The invention can therefore also be used in diagnostic kits and in creating immunogenic tolerance in e.g. food allergies.

Furthermore, we show that some sensitizing compounds (including, but not limited to, glyoxal, CDNB and dBBr) result in increased levels of acute-phase proteins such as serum amyloid P component (SAP). Normally, the plasma concentration of SAP increases in response to inflammation. It is thus likely that some sensitizing molecules instigate a perpetual inflammatory response. SAP is highly resistant to proteolysis and its binding stabilizes amyloid fibrils, enhances their formation in vitro, and contributes to their pathogenic deposition and/or persistence in vivo in amyloidosis; autoimmunity and Alzheimer's disease. It is known that chronic inflammation can lead to self attack (autotoxicity) due to the innate immune defence, and the important role of inflammation in Alzheimer's is getting more and more attention. SAP is directly neurocytotoxic, and it has also been suggested that SAP may cross the blood-brain barrier. SAP binds to keratin bodies in human skin as well as to isolated KIF aggregates in vitro. Furthermore, the amount of anti-SAP antibodies in SLE correlates with the disease activity. SAP can also be found in urine. The finding of an increased amount of SAP in the sera of mice exposed to some sensitizers but not others (including, but not limited to, oxazolone) can also be utilized as an additional diagnostic feature in in vitro tests.

The molecular understanding of what happens when compounds encounter epithelia and epithelial cells, i.e. the blebbing response and that keratins and other proteins released in blebs are key proteins involved, has made it possible to develop products for in vitro diagnostic kits for determining:
which compound(s) a patient is allergic to
which neoepitopes a patient is allergic to
which cryptic epitopes a patient is allergic to
what ailments that a particular patient risk in epithelial contact with compounds (for example orally, through ingestion, through inhalation, through skin contact, etc.)
the extent to which that patient has breached its self-tolerance, i.e. the risk of that patient developing autoimmune diseases
the risk of that patient showing DHRs
the risk of food allergies
the risk of developing autotoxic diseases

These represent major advancements in the field of personalized medicine and since the testing is performed in vitro (i.e. the patient submits a blood sample), it is an undemanding test for the patient. Furthermore, once the information from the diagnostic kit (or the equivalent, detailed information elsewhere in the application) is known, then the invention is also that immunogenic tolerance principles can be utilized to prevent/cure/alleviate the diseases (including but not limited to ACD, autoimmune diseases, DHRs, autotoxic diseases). This has not been possible before, as this detailed understanding of a particular patients status is only made possible through the findings presented herein.

In summary, all of the above applications are made possible through our new molecular understanding and thus represent applications that previous technologies are unable to provide.

To study the reactions occurring upon contact with epithelial tissue such as the skin, the inventors have deployed caged haptens (bromobimanes) that are non-fluorescent prior to displacement of the bromine atom. It should be noted that hazardous compounds that can cause immunological problems are called “haptens” in diseases such as allergic contact dermatitis. The usage of the word “hapten” should however not be taken to exclude all other conditions that can result through epithelial contact with compounds. On the contrary, the invention also includes using the knowledge obtained as a means to diagnose and treat/cure and/or alleviate other ailments including but not limited to autoimmune diseases and DHRs. For ethical reasons, the experiments have been performed on human skin excerpts, human cells (in vitro cultures) and mice. However, the present invention relates to all animals, including humans.

Uncaging of bromobimanes occurs preferably with thiols (cysteines) that are potent nucleophiles at physiological pH. Preferential reactivity towards thiols is advantageous since many, if not most; clinical haptens are thiol-reactive. The other major hapten-reactive moiety in peptides and proteins is the nucleophilic amine group (the —NH₂ group of lysines and the α-NH₂ group that constitutes the amino terminus of proteins.) A series of bimanes with none (synBim), one (mBBr), or two reactive groups (dBBr) was deployed to make major steps forward in elucidating the detailed molecular mechanisms behind sensitization and epithelial immunotoxicity.

The first step is that the molecules come into contact with the skin (the stratum corneum). Many of the reactive molecules react in the stratum corneum, something that can be seen in experiments with human skin excerpts and the caged (mBBr and dBBr) haptens. However, a significant proportion of the molecules penetrate further down to the suprabasal layers of the epidermis. It is likely that the penetration is due both to the acidic pH of the stratum corneum (the molecules react more slowly when the pH is low), but also because many of the available groups have reacted. In the suprabasal layers, the haptens have a hard time penetrating the cells, as the cornification of the cells creates a thick, relatively impermeable envelope around the suprabasal cells. Consequently, the haptens penetrate down to the basal layer. Here, the cell membrane is much thinner and more fluidic and the molecules can thus enter the basal cells. The experiments with human skin excerpts show, that in addition to the stratum corneum; cells in the basal layer are labeled by the caged haptens dBBr and mBBr.

The second step is that the molecules enter the basal cells. Since the extracellular environment is oxidizing, extracellular cysteines are mostly oxidized to disulfides. However, it is in the reduced, intracellular environment that the haptens find reduced thiols to react with. Once inside the cells the compounds will react with available (i.e. not sterically hindered) reduced cysteines (thiol groups). In this step, it is demonstrated that the labeled cells are basal keratinocytes. Furthermore, it was identified that one of the cysteines that the haptens react with is cysteine 54 (C54) of K5. Using a LTQ-FT-ICR mass spectrometer equipped with a nanospray source it was found that the thiol-reactive hapten mBBr modifies C54 of K5 in intact, full-thickness human skin and when living human keratinocytes have been exposed to mBBr. One of the reasons for intense reaction at C54 is presumably its high effective concentration just inside the cell membrane. It is vital in the life of an organism to fail-safe its epithelial integrity, and the bundling of the KIFs ensures a high effective local concentration of the peptide stretch containing C54, ensuring strong welding of the cell to its surroundings. Amino acids nearby in the primary sequence are consequently also located within physical reach of an incoming compound in similarly high effective concentrations. Looking at blebbing brought on by amine- and arginine-reactive compounds from this angle, the residues K71 and R72 are the closest candidates in the primary sequence.

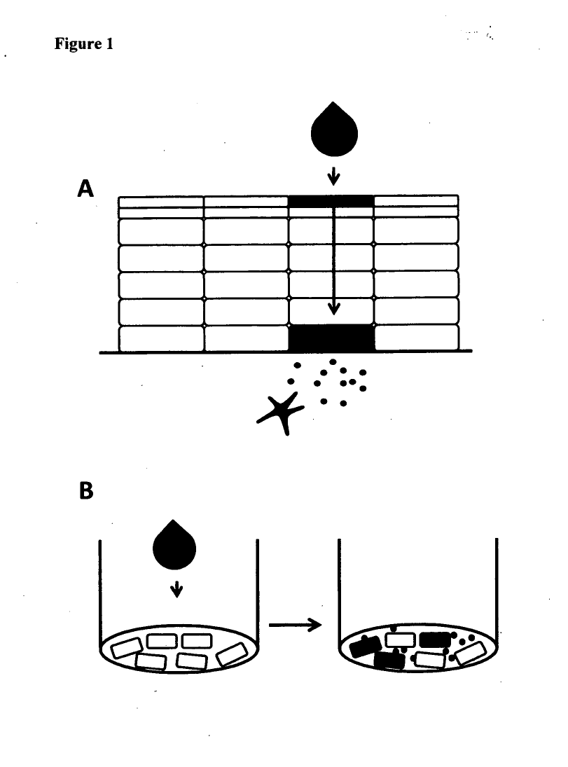

The third step is that this residue (C54) is situated in a stretch of K5 that has been shown through its interactions with desmoplakin, to be vital in maintaining the integrity of the desmosome. It is known that epithelial cells with a compromised cell-cell (and/or cell-matrix) contact undergo apoptosis (“anoikis”). The blebbing that is the result of contact with sensitizing compounds seem to share at least some mechanisms with the naturally occurring apoptosis. Basal keratinocytes consists of up to 35% of insoluble keratin intermediate filaments (KIFs). As basal keratinocytes occasionally undergo apoptosis, a mechanism of waste removal of the debris of insoluble keratins has evolved. In the body, apoptotic basal cells package their remnants in so called “keratin bodies”, that have been shown to consist to a large degree of K5 and K14. The keratin bodies bleb off the cells. Thus, when the keratinocytes are exposed to sensitizing compounds, at least some of the mechanisms behind the naturally occurring apoptosis happen, and KIFs (and other cellular remnants) are packaged in blebs. This step is thus that haptenation provokes the cells to produce fluorescent, bimane-containing blebs (FIG. 1). This did not occur when the non-reactive, non-allergenic substance synBim was added to the cells. Non-fluorescent, clinically relevant haptens such as oxazolone (4-Ethoxymethylene-2-phenyl-2-oxazolin-5-one), formaldehyde, CDNB (1-Chloro-2,4-dinitrobenzene), glyoxal, etc. also provoke a blebbing response. Through western blot experiments using K5 and K14 antibodies, it was shown that the blebs resulting from addition of predominantly amine-reactive (e.g. oxazolone) and predominantly arginine-reactive (e.g. glyoxal) also contain keratins.

In SDS-PAGE experiments it was shown that thiol-, amine-, and arginine-reactive sensitizers give rise to blebs that contain the same protein bands. In this step, it is thus shown that by adding haptens to keratinocytes, the haptens induce expulsion of a high amount of cryptic epitopes that are normally concealed from the immune system through their intracellular location and/or neoepitopes consisting of haptenated/differently processed peptides and proteins. It was also shown, using a LTQ-FT-ICR mass spectrometer coupled to a nanospray source, that the blebs contain other proteins, for example peptidyl-prolyl cis-trans isomerase A, stathmin, calmodulin, cofilin-1, transgelin-2, 14-3-3 protein sigma, annexin A2, tubulin, keratin 6A 6B 6C, transitional endoplasmic reticulum ATPase, endoplasmin, and importin-5. Furthermore, it was found that neoepitopes (hapten-protein conjugates) are expelled (as exemplified by the verification of the C54-hapten conjugate in the blebs). It was also shown that the sensitizing compounds also modify other proteins and that these proteins are released in blebs, as visualized by fluorescent bands on SDS-gels. The neoepitopes concept is further corroborated by the results that bidentate haptens (such as dBBr) cause cross links between for example keratins.

In addition to the formation of neoepitopes, other problems ensue from cross linking such as altered protein conformations/processing/clearance. In line with this theory, it can be seen that some sensitizing compounds (including, but not limited to, dBBr, CDNB and glyoxal) result in increased levels of acute-phase proteins such as serum amyloid P component (SAP). Normally, the plasma concentration of SAP increases in response to inflammation. It is thus likely that some sensitizing molecules instigate a perpetual inflammatory response. SAP is highly resistant to proteolysis and its binding stabilizes amyloid fibrils, enhances their formation in vitro, and contributes to their pathogenic deposition and/or persistence in vivo in systemic amyloidosis, autoimmunity and Alzheimer's disease. It is known that chronic inflammation can lead to self attack (autotoxicity) due to the innate immune defence, and the prominent role of inflammation in Alzheimer's is getting more and more attention. It has also been suggested that SAP may cross the blood-brain barrier, and it is directly neurocytotoxic. SAP has been found to bind to keratin bodies in human skin as well as to isolated KIF aggregates in vitro. Furthermore, the amount of anti-SAP antibodies in SLE correlates with the disease activity. SAP can also be found in urine. The finding of an increased amount of SAP in the sera of mice exposed to some sensitizers but not others (including, but not limited to, oxazolone) can be utilized as an additional diagnostic in vitro feature, studying e.g. different aspects of autoimmune diseases. Some sensitizing compounds (including, but not limited to dBBr, oxazolone, glyoxal and CDNB) result in increased levels of antinuclear antibodies (ANA). The presence of elevated levels of ANA is a commonly used marker for autoimmune diseases such as SLE, RA and SS.

The fourth step is that it has been shown that in the body, keratin bodies drop below the basement membrane. It has been shown that keratin bodies are devoured by antigen-presenting cells. In the dermis, these are chiefly the dermal dendritic cells. Epidermal antigen-presenting cells such as the Langerhans cells are of course also exposed to these cellular remnants, and these cells are in all likelihood also part of the sensitization process. In this step, the neo- and cryptic epitopes are thus devoured by antigen-presenting cells. To demonstrate that keratins are released and function as antigens, ELISA capture of autoantibodies has been performed against keratin in mice that have been exposed, through skin contact, to haptens. The results show that haptens expose keratin to the immune system, and that the protein fragments are antigenic since mice develop autoantibodies against them. This also corroborates that other, less abundant proteins that were found in the blebs (e.g. transitional endoplasmic reticulum ATPase and calmodulin) are released in the body upon contact with sensitizers.

These steps are true also for haptens that preferentially react with other amino acids than cysteine. This was shown by adding a panel of haptens to human keratinocytes, and visualizing the cellular response. Haptens, as opposed to the vehicle (DMSO) instigate blebbing. Non-allergenic compounds such as synBim or nonanoic acid do not induce blebbing. The irritant compound SDS does not cause blebbing, instead the cells seem to “dissolve” when exposed to SDS.

The blebs contain keratins and other proteins. That the blebbing is a general response that also occurs in the body has been demonstrated by capturing autoantibodies against K14 in mice that have been exposed to sensitizing compounds directed at diverse nucleophiles (amino acids) that react through different reaction mechanisms with the same nucleophiles and that are monodentate or polydentate.

These steps 1-4 establish sensitization. In vitro diagnostic tests and immunological tolerance based on these discoveries is the core of the invention herein.

Through a number of experiments, it was thus found that prominent targets in human tissue are keratinocytes. It is surprising that, even though the epidermis consists of ˜95% keratinocytes or remnants (corneocytes) thereof, the role of keratinocytes has been overlooked. The stratified nature of the skin allows haptens to reach basal keratinocytes that contain large amounts (up to 35% of the total protein content of the cells) of keratins 5 and 14 (K5 and K14).

The invention is thus based on the discovery that keratinocytes that have been subjected to compounds that are sensitizers, produce blebs, and that these blebs contain proteins such as keratins that play an important role in establishing immunity. Other less abundant proteins, also found in the blebs (e.g. stathmin and calmodulin), are also released in the body upon contact with sensitizers. The utilization of different aspects of the proteins in the blebs is thus the basis of the invention. Some sensitizing compounds give rise to increased levels of acute-phase proteins such as serum amyloid P component (SAP) and it is the intention that this is also a part of the invention.

Basal cells in stratified squamous epithelia express K5 and K14 in high abundance, up to 25-35% of the cellular content. The described mechanisms (steps 1 through 4, vide supra) are therefore also likely to play a role in ailments whereby sensitization and elicitation is independent of the route of entry of the substance into the body such as through ingestion, eye contact, through inhalation, etc. Thus, if a compound causes blebbing through skin contact there is a high risk that the compound in question also can induce other conditions besides allergic contact dermatitis. These conditions include chemically induced asthma but do not exclude other ailments. The proteins in the blebs can therefore also be utilized in tests for other conditions that can be caused by epithelial contact with a compound. Proper measures can then be undertaken to reduce future risks. The information obtained from these in vitro diagnostic kits can also be used to alleviate symptoms, and/or treat, and/or reduce the risk of experiencing allergies through immunological tolerance principles.

Furthermore, the finding that the identical or highly homologous epitopes are encountered irrespective of the route of entry of the compound into the body (exemplified by K5/K14 and K8/K18) has other implications. An example of this is DHR where the blebbing response also could be important. Another object of the invention is therefore to use the proteins in the blebs to gauge the risk of that particular patient's risk of experiencing drug-induced hypersensitivity reactions (DHRs). Proper measures can then be undertaken to reduce future risks. The information obtained from these in vitro diagnostic kits can also be used to alleviate symptoms, and/or treat, and/or reduce the risk of experiencing DHRs through immunological tolerance principles. The present invention thus represents a major advancement in predictive screening for adverse drug reactions upon epithelial contact.

It is therefore proposed that once blebbing is instigated, and the immunological threshold is passed, self-tolerance is breached. These breaches of tolerance include several targets including K5, K14, and epitopes spreading through homology between released proteins and other self-proteins (for instance between K5 and K8 and K14 and K18). Thus, the invention presented herein, can estimate to which extent a particular person has breached his/her self-tolerance and thus estimate the risk of downstream autoimmune diseases. Proper measures can then be undertaken to reduce future risks. The information obtained from these in vitro diagnostic kits can also be used to alleviate symptoms, and/or treat and/or reduce the risk of experiencing autoimmune diseases through immunological tolerance principles.

It can thus be concluded that sensitizing compounds, in general, prompt keratinocytes to produce blebs. This corresponds to a key gate in a logistic chain of events that ultimately leads to sensitization (ACD); and/or autoimmune diseases; and/or other allergies; and/or primes the individual for DHRs and/or leads to autotoxic events. The specific outcome in each case is likely to depend on the immune system of the individual and his/her environmental cues.

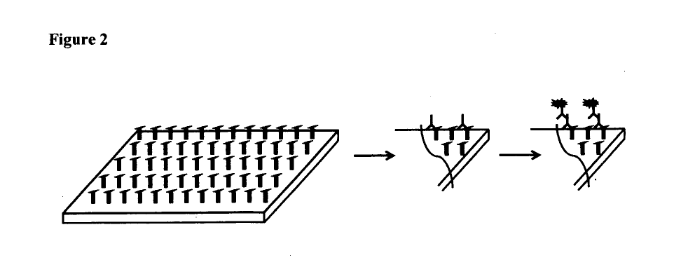

As was previously mentioned, the invention presented herein includes the use of keratins and other proteins, including proteins found in the blebs (for example peptidyl-prolyl cis-trans isomerase A, stathmin, calmodulin, cofilin-1, transgelin-2, 14-3-3 protein sigma, annexin A2, tubulin, keratin 6A 6B 6C, transitional endoplasmic reticulum ATPase, endoplasmin, and importin-5), and/or peptides with amino acid sequences based on these proteins. Such proteins and peptides are hereinafter included in the term amino acid containing epitope targets. The term amino acid containing epitope target further encompasses mutations thereof and also homologues from other species. This term also includes peptides with sequences based on those proteins, proteins and/or peptides with modifications (e.g. peptides with attached haptens), homologous proteins and/or peptides with or without haptens, protein/peptide epitopes that are the result of epitope spreading, peptidomimetics and/or other more drug-like molecules that share the necessary interactions between these epitopes. In addition, the term may also include proteins whose expression is altered due to the action of epithelial encounter (including, but not limited to SAP and ANA). The amino acid containing epitope targets are the basis of arrays and libraries (see schematic illustration of this in FIG. 2). The term array will be used to designate a collection of amino acid containing epitope targets/etc. The samples from the patients (e.g. blood/serum) are run over the array, and analyzed with respect to what antibodies/T-cells are present in the sample.

Array design: The arrays will be designed using scientific knowledge of what constitutes a “good” or likely epitope (or not). That is, in most cases, the entire proteins will not be covered. The presence of haptens will also be taken into account in the design of the epitopes, as covalent attachment of a hapten in the majority of cases significantly alters the chemical properties of the amino acid that becomes derivatized. Cross-linking events (in the case of bidentate haptens or haptens that oxidize for example cysteines to disulfides) that create neoepitopes will also be considered in the design and construction (synthesis) of the arrays. Neoepitopes formed from unnatural (due to the presence of a hapten) processing of proteins/peptides can be in the design and construction (synthesis) of the arrays. In these arrays, the peptides/proteins are portioned out in a coded fashion so that the exact position of each peptide/protein is known. The proteins/peptides present in the arrays can for example be obtained through recombinant principles or through direct preparation and purification from cells or animals. The peptides can be synthesized by e.g. solid phase peptide synthesis, either directly on plates (or the equivalent) or separately and then spotted (or the equivalent) onto plates. There is a multitude of commercially available and publically known ways to approach this.

Haptenated epitopes: In order to identify haptenated epitopes, an array of haptenated proteins/peptides will also be analyzed. This can be performed in solution or on solid support either before spotting or on the plate (or equivalent). In some occasions, haptens might be attached prior to cleavage of the protecting groups on the peptides (during peptide synthesis), but for the majority of haptens, derivatization (i.e. reaction between hapten and peptide/protein) will occur post cleavage of the protecting groups. Again, there are many commercially available and publically known ways to approach this. The controlled construction of the arrays, and the fact that the haptens are attached to the proteins (i.e. rendered “unreactive”) will alleviate the problems of using sometimes relatively unstable molecules in the formulations applied during patch testing. For bidentate haptens (i.e. haptens that link two parts of a protein or two different proteins), cross-linking between peptides or proteins can be performed in solution and/or on solid support depending on the hapten in question. For pro- and prehaptens, activation steps will be included prior to coupling. Alternatively, the activated molecules will be synthesized directly and coupled to the peptide/protein. In many cases, there is a high likelihood that the exact covalent linkage between the hapten and the amino acids (of the protein/peptide) is not involved in the immunogenic response to a large degree. That means that in some cases, it might be more desirable to couple the moiety (i.e. the hapten part) that is recognized by the binders (antibodies and/or T-cells) to its carrier protein/peptide through a covalent link that is better suited for the application. Better suited could mean easier coupling to the carrier, generating a more stable covalent link that survives during storage of plates (or the equivalent) and during the assay conditions, a more soluble hapten-derivative, easier synthesis of activated hapten-derivative prior to coupling, etc. Also, a battery of protected “unnatural” amino acids can be pre-assembled from the list of known haptens and their reactivity patterns (e.g. thiol, amine-, arginine-, hydroxyl-reactive, etc.). These pre-haptenated, protected amino acids are then ready for the assembly line in peptide synthesis. Of course, post-translational events and stress-responses such as for example phosphorylations can be included in the design and assembly of the peptide libraries using commercially available (or custom made) amino acids.

High throughput format: The arrays and libraries can be made in high throughput formats. This includes formats such as microtiter plates, microfluidic and nanofluidic chips, CD laboratories such as those from GYROS™, etc, but do not exclude other formats. Through the use of standard (or customized) lab robots, a very large number of plates can be analyzed in parallel. For each patient/customer, many plates can be analyzed. Thus, and in contrast to today's technology (patch testing), this enables simultaneous screening of all known haptens. The individual simply submits a blood-derived sample (e.g. serum). The sample is subjected (if necessary) to clean up, run over the array by procedures known to a person skilled in the art, and analyzed with respect to antibodies and/or T-cells that bind to the arrayed targets. Antibodies and T-cells will collectively be called “binders”. This corresponds to a procedure called epitope mapping.

Signaling the binding event (the output signal): Once the binders in the samples bind their epitope target on the array, the binding event will be signaled. This is a common situation in the lab and very many solutions to the problem exist. Examples include the use of detection antibodies (that bind the “binders”) carrying fluorescent probes/biotin/enzyme substrates etc., other binders such as molecules from the AFFIBODY® platform or DNA and/or RNA molecules that bind the binders and signal the binding through e.g. fluorescence, sandwich ELISA formats, surface plasmon resonance, etc. as known in the art. Quantitative results can be obtained by comparing the signal from the unknown sample (the sample from the patient) in parallel with a standard series. The standards (duplicates or triplicates) are run with each assay to ensure accuracy. The output of the in vitro diagnostic kit can thus be measured using various methods, including but not limited to:
i. Changes in fluorescence upon binding. A plate (or the like) is coated with an array of amino acid containing epitope targets. The plate is blocked and the diluted blood-derived samples (e.g. serum) are added as known in the art. Any antibodies in the samples bind to their respective epitope/target. The plate is washed and the presence of antibodies is detected by the addition of a detection antibody (or binder that provides the same function) carrying a fluorescent probe. The amount of fluorescence at a specified wavelength correlates with the amount of antibodies present in the serum sample. The presence of antibodies to the target epitope implies that the patient is allergic to that epitope/target.
ii. Enzyme-linked detection upon binding (ELISA methods). A plate (or the like) is coated with an array of amino acid containing epitope targets. The plate is blocked and the diluted blood-derived samples (e.g. serum) are added. Any antibodies in the samples bind to their respective epitope. The plate is washed and antibodies in the serum samples are detected by the addition of a detection antibody (or binder that provides the same function). The plate is again washed, and a secondary binder that binds the detection antibody is added. The secondary binder is linked to an enzyme (for example horse radish peroxidase, HRP or alkaline phosphatase, ALP). In the final measuring step, enzyme substrates that form chromogenic end products are added. The chromogenic end products then correlate with the amount of antibodies present in the sample. This procedure is performed as is known in the art. The presence of antibodies to the target epitope implies that the patient is allergic to that epitope/target.
iii. Quartz crystal microbalance techniques (including but not limited to the Q-SENSE™ platform) that shows when proteins (i.e. serum antibodies) coat the surface of the sensor that has been pre-derivatized with epitopes.
iv. Surface plasmon resonance techniques (including but not limited to the BIACORE™ platform). This is a type of label free interaction analysis, and the extent to which antibodies in the sera interact with their partners (i.e. the epitopes) immobilized on a sensor surface is signaled Again, the presence of antibodies to the target epitope implies that the patient is allergic to that epitope.
v. The proliferative capacity of T-cells to various stimuli in vitro can be measured using peripheral blood mononuclear cells (PBMCs) from whole blood or Ficoll-Isopaque separated blood. The output signal can for example be based on a radioactive assay (incorporation of [³H]TdR); by flow cytometric assessment of surface molecule expression on T cells (including, but not limited to CD38, CD4, CD8); or various enzyme-linked immunosorbent assays (ELISAs). Examples include EPISCREEN™ T cell epitope mapping platform from Antitope Ltd (Cambridge, UK), and CD8⁺ T Cells: PRO5® MHC Class I Pentamers and/or CFSE T Cell Proliferation Assays both from ProImmune (Oxford, UK). The epitope mapping services supplied by a large number of companies can also be used (including, but not limited to CD8® T Cells: REVEAL™ & PROVE® Class I, and CD4⁺ T Cells: REVEAL™ Class II) by ProImmune (Oxford, UK).
   Information obtained from the sample: The in vitro diagnostic kit of our invention will provide information on:
The epitope targets, i.e. precisely which part of the proteins that are recognized by the binders. This gives leads regarding the risk of epitope spreading through homologies, etc. This information is also used as a guide in personalized immunological tolerance treatments (see below). This represents a major breakthrough, since no epitopes can be detected through patch testing.
The specific interaction with haptens (i.e. haptenated epitopes). In contrast to the current diagnostic method (patch testing), the present invention provides the patient/customer with objective diagnostic information. Knowing which compounds that are specifically recognized (together with the carrier proteins) means that the patient can avoid the compounds in question.
A complete list of haptens and any cross-reactive haptens, i.e. haptens that are analogues to the recognized hapten and that also give rise to symptoms on exposure. This represents a major breakthrough compared to today's technology, where only a limited number of haptens that can be tested. Through the present invention, the risk of missing one or more allergens is vastly reduced.
If there are, or is a risk of allergies contracted through epithelial contact (i.e. orally, through skin contact, eye contact, through inhalation, etc.) with other compounds. A major breakthrough made possible through the understanding that (in some cases), the same mechanisms operate behind epithelial exposure, irrespective of the route of entry into the body.
If there is a breach of self-tolerance, i.e. if autoantibodies and/or T-cells against self-proteins are present. This indicates risk that the patient has in developing autoimmune diseases/risk of DHRs caused by epithelial contact with drugs/etc. Again, a major breakthrough made possible through this understanding of the mechanisms behind epithelial exposure to different compounds.
If there is a risk of epitope spreading, i.e. if epitopes present in other proteins are recognized. This can have major implications as this can lead to for example autoimmune diseases, food allergies, etc.
Furthermore, and in contrast to the present technology, the test of the invention allows for a detailed and objective health record to be kept. Thus, progression of allergies and/or other ailments can be monitored by tracking antibodies/T cells, etc. as well as epitope spreading, etc. This provides the doctor and the patient the possibilities of keeping the diseases in check and/or follow treatment and/or forewarn the emergence of other diseases (for example autoimmune diseases, DHRs, autotoxic diseases, etc.).
The presence of other proteins such as for example Serum amyloid P component (SAP) and antinuclear antibodies (ANA) also aids in guiding the patient and allows the doctor to advise the patient in his/her life decisions.

Controls, quality assurance, and quantitative/qualitative outputs: As in all tests, positive and negative controls have to be included to ensure proper day-to-day quality of the tests. Positive controls could for example constitute fluorescently labeled antibodies/T-cells raised against the sequences with/without haptens. As for negative controls and/or background checks, these could for example constitute spots with “dummy peptide sequences” with no relation to known sequences. Normal, standard levels of antibodies/T-cells are derived from healthy humans with no contact allergies. The analysis can be qualitative providing yes/no answers, or quantitative providing information regarding the titer of binders (e.g. antibodies and/or T-cells). The titer is a measure of the amount of binders present in the sample. Both of these are guides to personalized medical advice. The information obtained is also a guide for treatment through immunogenic tolerance principles (see below).

The diagnostic method could thus includes the following steps: blood sampling from patient, coagulating to purify the serum or centrifuging the blood to get the plasma according to standard methods. These first steps are performed at the clinic. The samples are then subjected to clean-up if needed to remove remaining assay-interfering hemoglobin before diluting the samples and applying them on the chip coated with peptides and/or haptenated peptides.

The diagnostic kit for ACD/Autoimmune diseases/DHRs includes one or several of the following components: a chip or plate (or another format suitable for high throughput analysis) coated with amino acid containing targets with or without haptens (the targets include, but is not limited to, e.g. K5 and K14 peptides (and other proteins found in the blebs), clean-up cocktail, control samples, blocking buffer, binding buffer, washing buffer, secondary antibody conjugated with a fluorescent molecule (if fluorescence is used for detection) or with HRP/ALP (if ELISA is used as a detection method). If any other detection method is recommended, such as Surface Plasmon Resonance, quartz crystal microbalance techniques or equivalent, the customer can send the chip or plate for analysis by a professional laboratory. This kit can also be used to estimate to what extent the patient has breached his/her self-tolerance and the risk of that patient showing DHRs. To diagnose allergy contracted through other epithelial tissue such as the pulmonary tissue, the chip or plate is coated with K8 and K18 peptides and/or haptenated K8 and K18 peptides in addition to K5, K14 and other bleb proteins. The kit can also be used for T cell epitope mapping. PBMCs are isolated from a subject's blood and defibrinated according to procedures known to a person skilled in the art. The T cells are then incubated on the plate (or another format suitable for high throughput analysis) with the epitopes. Tritiated thymidine is added to the cell cultures and incorporated thymidine (i.e. T cell stimulation and proliferation) in each well is measured in a scintillation counter and compared to unstimulated T cell cultures. For T cell epitope mapping, the kit may e.g. include a chip or plate (or another format suitable for high throughput analysis) coated with amino acid containing epitope targets, cell culture medium and normal T cell populations.

There is no cure for ACD today. Avoiding the compound in question, and any cross-reactive compounds, is the only option. The findings presented herein fortunately also open up a solution in treatment of ACD. Once the detailed information from the diagnostic kit (or the equivalent detailed information obtained elsewhere) is known, then the invention is also that immunogenic tolerance principles can be utilized to alleviate and/or prevent the diseases (including but not limited to ACD, autoimmune diseases, DHRs). This constitutes a form of treatment/cure, something that has not been possible before, as this detailed understanding of a particular patient's health status and risks is only made possible through the findings presented herein.

Furthermore, through the described in vitro diagnostic kits (and other information) major targets of the immune system are identified and a “vaccine” can be based on these targets. A vaccine concerns naïve individuals, i.e. individuals that have not developed antibodies/T-cells against the target(s). Thus the invention deals with making use of immunological tolerance principles. By using these principles, tolerance to known, common antigens can be created by manipulating the immune system, creating tolerance to the chemical allergen in question. If the allergen is identified as a haptenated protein, where both the hapten part and the carrier protein is part of the epitope, several problems arise. First, it is not ethical to administer the reactive compounds themselves as a small compound can cause much harm. Second, the compound will not magically only react with the desired proteins, but it will react with whatever is available. However, a “dead” compound that is already coupled to its primary protein/peptide target(s) or mimics thereof is much more harmless and it constitutes the antigen in question. Prior to the detailed understanding presented herein, the key protein targets have not been known, and this approach has consequently not been possible before.

The invention is thus also to induce tolerance by immunological tolerance principles. This includes for example repeated administration of large doses of antigen, or of small doses that are below the threshold required for stimulation of an immune response. A specific form of acquired tolerance is oral tolerance. Oral tolerance has evolved to treat external agents that gain access to the body via ingestion. One example of the invention is thus to administer the allergen(s) orally.

There are no vaccines against ACD today. Furthermore, there are no ways to cure ACD. Once sensitized, an immunological memory is created, and the only way to avoid the symptoms is henceforward to avoid the compound and any cross-reactive compounds. This has major socioeconomic costs, as ACD is a common occupational hazard and more than 15% of the population in the Western world is sensitized to one or more compounds.

Some experiments on creating tolerance towards haptens have been performed, for example to oxazolone and nickel using experimental animals, notably naïve mice. Naïve means that the animal has not been exposed to the hapten before, and this is thus a type of “vaccination” against the hapten. This has resulted in some protection against the hapten, but it is an approach that cannot be carried over to humans (see below).

There are no cures for ACD today. However, it would be highly beneficial to be able to cure ACD, both from the standpoint of the increased quality of life of the affected individual, and to the decrease in sick days. Today, desensitization (hyposensitization) is a treatment in which the patient is gradually vaccinated with progressively larger doses of the allergen (for example grass pollen) in question. However, this approach has not been applicable in the case of ACD, as the “allergens” (i.e. hapten-protein complex, cryptic epitopes, neoepitopes, etc.) have not been known prior to the disclosure presented herein.

The immunotherapy approaches that are most common are subcutaneous injection and oral (sublingual) administration, the latter being preferred through the more restricted severity of the side effects. Allergen immunotherapy can reduce the need for medication/reduce the severity of the symptoms/eliminate hypersensitivity altogether. Allergen-specific immunotherapy thus offers substantial advantages compared to other therapies that merely suppress the symptoms.

Towards vaccines/treatments/cures for ACD: Even though feeding mice with haptens (small molecules) has resulted in some protection, that approach cannot be carried over to humans. It is simply unethical to “feed” human beings with small, often reactive, molecules (haptens) that will react with all types of biomolecules. This causes uncontrolled toxicological, carcinogenic and/or mutagenic effects. Furthermore, if the hapten (small molecule) does not react, it cannot evoke tolerance principles, but it can still cause unwanted pharmaceutical effects. However, by coupling the small compounds (i.e. haptens) to a carrier protein/peptide or mimic thereof, two things are accomplished. The first is that the resulting conjugate can be administered since the haptens are “deactivated” through the coupling procedure, and therefore no longer reactive. This means that the uncontrolled reactions are prevented and thus the unwanted toxicological, carcinogenic and/or mutagenic effects are thus avoided. The second is that the antigen is formed through the reaction between the hapten and its known biological target (the carrier). This is crucial, since the antibodies and/or T-cells recognize parts of the carrier protein/peptide as well as the hapten (in applicable cases). In order to create tolerance towards the antigen that is released in the body of the patient in his/her day-to-day life, the correct carrier should be employed.

It has been shown that keratins are major targets for haptens. Since 95% of the cells in the epidermis (our major epithelial barrier) are keratinocytes, and between 25 to over 80% of the protein content of those cells are keratins, keratins play a major role in establishing immunotoxicological reactions (ACD, chemically-induced asthma, autoimmune diseases, etc.). It has also been shown that C54 of K5 is covalently modified by thiol-reactive haptens in human skin and human cells (keratinocytes). Furthermore, it has been shown that this allergen is expelled from living human keratinocytes in blebs. It has also been shown that keratins are major carrier proteins, as demonstrated by autoantibodies in murine sera from mice that have been treated with haptens on their skin. The present invention makes it possible to develop products that have the haptens coupled to the correct carrier protein(s) in the cases where the hapten is part of the immune response, and without hapten in the cases the immune response is targeted to cryptic/neoepitopes released through hapten exposure. Since the major carrier proteins/peptides have been unknown prior to the presented findings, the present disclosure represents a major technological and medicinal breakthrough. The invention represents a major advancement in the possibilities of creating tolerance (either in naïve individuals or individuals that are already sensitized to one or more compounds) towards the antigens in ACD, DHRs and other allergies contracted through epithelial contact with various compounds.

The term “carrier” is used to designate biomolecules that, either together with the hapten or not, form the antigen to which the patient is allergic. As was previously mentioned, the invention presented herein includes the use of keratins and other proteins found in the blebs as carrier proteins. The proteins/peptides are the carriers to which the hapten(s) are coupled prior to administration to the patient, if the patient is sensitized to the hapten moiety. What amino acid containing epitope target to use to induce tolerance is determined from the epitope mapping result for that individual, i.e. the target with the most intense antibody or T cell response will preferably be used.

Immunogenic tolerance can be made to happen through several routes of administration of the antigen, and the invention covers all possible routes of administration. One special case of tolerance is oral tolerance. Here, powerful tolerance mechanisms are in place to deal with the large number of foreign proteins and antigens we encounter on a daily basis through eating and drinking.

It is possible that the carrier need not be a protein or peptide but a mimic thereof. A peptidomimetic, peptide with non-natural stereochemistry, etc. can be more stable in vitro and/or in vivo and it can for that reason be a better choice. It could have, or not have, a hapten attached, depending on the actual allergen. The mimic could potentially be even further removed from a traditional biomolecule through the use of more drug-like carriers; provided that tolerance principles are still evoked.

If the hapten is part of the antigen, then hapten-carriers complexes need to be synthesized and administered. The same principles apply for synthesis as those detailed above in the description of synthesis of hapten-peptide conjugates in the in vitro diagnostic section of the invention. Notably, the linkage between the hapten and its carrier need not be what would actually be the case in vivo, provided that tolerance principles are still evoked. The in vivo stability of the hapten-carrier complex needs to be addressed, such that the conjugate is not broken down prematurely.

It is also possible that the entire complex and/or the autoantigen can be substituted for a more drug-like compound with better pharmacokinetics, etc., provided that the mimic elicits protection against sensitizers/autoantigens/etc. Even though it is beneficial to know the exact epitope pattern for a particular individual, immunogenic tolerance principles can also be invoked to “common targets”. These are for example keratins with/without haptens, as keratins constitute such a large percentage of protein in epithelia such as the skin; however other epitopes are not excluded. That is, haptenated (or without hapten) keratin-derived peptides/proteins/peptidomimetics/amino acid containing epitope targets/etc. can be e.g. orally administered to generate oral tolerance according to standard immunological tolerance principles. This can also be done in a preventive treatment, i.e. “vaccination”. One application is targeted vaccination of certain workgroups e.g. hairdressers, where ACD is a very common occupational hazard.

Immunogenic tolerance—treatment/vaccines for diseases caused by epithelial contact with compounds: The invention can also be used for tolerance towards autoantigens/antigens in allergies contracted through contact between a compound and epithelial cells (oral, eyes, through inhalation, etc.)/DHRs/etc. This is based on our findings that sensitizers, in general, provoke keratinocytes (epithelial cells) to produce blebs that contain cryptic epitopes/neoepitopes. As was previously mentioned, the invention presented herein includes the use of keratins and other proteins associated with the blebbing response (including for example keratin 1, keratin 5, keratin 8, keratin 10, keratin 14, keratin 18, peptidyl-prolyl cis-trans isomerase A, stathmin, calmodulin, cofilin-1, transgelin-2, 14-3-3 protein sigma, annexin A2, tubulin, keratin 6A 6B 6C, transitional endoplasmic reticulum ATPase, endoplasmin, and importin-5). Thus, the invention is that the same type of principles as detailed above in the treatment and prevention of ACD are applicable also in these cases.

All parts of the invention can be used for, but is not limited to, humans, nonhuman primates such as chimpanzees and other apes and monkey species, farm animals such as cattle, sheep, pigs, goats and horses, domestic mammals such as dogs and cats, laboratory animals including rodents such as mice, rats and guinea pigs, and the like. Adult and newborn subjects, as well as fetuses, whether male or female, are intended to be covered in the invention. In preferred embodiments, the subject is a mammal, including humans and non-human mammals. In the most preferred embodiment, the subject is a human.

### EXAMPLE 1

Diagnosis of ACD/Autoimmune Diseases/DHRs/etc. Using Blood-Derived Serum from Hapten-Exposed Mice

### Epithelial Exposure of Various Compounds and Collection of Sera

Mice: CBA/Ca, females, 8-12 weeks old at the start of the experiment. (BK Scanbur, Sollentuna, Sweden).

Dissolve the chemical to be tested in molecular biology grade dimethyl sulfoxide (DMSO, Sigma Aldrich, Stockholm, Sweden) or other solvent recommended by OECD (e.g. acetone:olive oil 4:1) to yield the desired concentration.

For extreme sensitizers (EC3<0.1%): use a concentration 10 times the EC3-value. For strong sensitizers (0.1%<EC3<1%): use a concentration 10 times the EC3-value. For moderate sensitizers (1%<EC3<10%): use a concentration 2 times the EC3-value. For weak sensitizers (EC3>10%): use the same concentration as the EC3-value.

Examples of Compounds Used in Experiment without Booster Dose on Day 10:

Extreme sensitizers: Oxazolone (4-Ethoxymethylene-2-phenyl-2-oxazolin-5-one, 1.4 mM) CDNB (1-Chloro-2,4-dinitrobenzene, 7.4 mM), p-Benzoquinone (9.3 mM); Strong sensitizers: mBBr (44.3 mM), dBBr (48.6 mM), qBBr (48.9 mM), p-Phenylenediamine (148.0 mM), Benzyl bromide (116.9 mM), Formaldehyde (2.3 M); Moderate sensitizers: 1,2-Benzisothiazolin-3-one (1.52 M), Phenylacetaldehyde (2.5 M), α-Methyl-cinnamicaldehyde (3.08 M), Dihydroeugenol (2-Methoxy-4-propylphenol, 4.09 M); Weak sensitizers: α-Hexyl-cinnamicaldehyde (5.55 M), Eugenol (2-Methoxy-4-(2-propenyl)phenol, 7.82 M), Benzyl benzoate (8.01 M), 1-Butanol (2.7 M), Phenyl ethyl alcohol (4.1 M); Irritant: SDS (Sodium dodecyl sulphate, 346.8 mM). DMSO is used as control. All chemicals are purchased from Sigma Aldrich (Stockholm, Sweden).

Examples of Compounds Used in Experiment with Booster Dose on Day 10:

Extreme sensitizers: Oxazolone (4-Ethoxymethylene-2-phenyl-2-oxazolin-5-one, 1.4 mM) CDNB (1-Chloro-2,4-dinitrobenzene, 7.4 mM)); Strong sensitizers: dBBr (48.6 mM), Glyoxal (ethane-1,2-dione, 1.27 M). DMSO was used as control. All chemicals are purchased from Sigma-Aldrich (Stockholm, Sweden).

### Procedure:

Apply 25 μl of the solution to the back of both ears (25 μl on each ear), once daily, on day 1, 2 and 3. Apply a booster dose on day 10 (if necessary). Sacrifice the mice on day 6 (or day 13 if a booster dose is used) by anesthetization with isoflurane (Kent Scientific Corporation, Torrington, Conn., USA) followed by inhalation of carbon dioxide (AGA, Lidingö, Sverige). To collect blood: Use a 1 ml syringe (B. Braun Medical Inc., Bethlehem, Pa., USA) with a 0.4 mm×12 mm injection needle (B. Braun Medical Inc., Bethlehem, Pa., USA). Place the tip of the needle just below the breastbone so that the syringe is linear to the abdomen. Insert the needle just below the breastbone and press it towards the heart. When inside the heart, withdraw blood. It is important to loosen the piston a little from the bottom of the syringe before inserting into the mouse. Carefully rotate the syringe during withdrawal to avoid clotting.

The blood is allowed to clot at room temperature for 30 minutes, then centrifuged at room temperature for 10 minutes at 2000 rpm using a Mikro 200R centrifuge (Hettich Zentrifugen, Tuttlingen, Germany). The serum is carefully pipetted off (using an automated pipette) and stored at −80° C. until use. Aliquot the samples to avoid excessive freeze-thaw treatment.

### ELISA—Detection of Anti-K14 Antibodies in Sera

All chemicals and reagents are from Sigma Aldrich, Stockholm, Sweden, unless stated otherwise.

### Coating

K14 (Genway Biotech Inc., San Diego, Calif., USA) is diluted in PBS to 0.5 μg/ml. 50 μl of the solution is added to half of the wells in a 96-well plate. The plate is covered with parafilm and incubated in a moist chamber at 4° C. overnight.

### Running

Each well is aspirated and washed with 300 μl wash buffer (TRIS, 0.05% TWEEN) for a total of three washes. After the last wash the remaining wash buffer was removed by invert the plate to clean paper towels. 150 μl blocking solution (0.5% BSA, 0.05M TRIS, pH 7.4) is added to each well and the plate is incubated at room temperature (RT) for 1 hour. The washing steps are performed four times as described earlier.

Each mouse serum sample is serially diluted 1:100, 1:5, 1:5, 1:5 in sample buffer (0.05M TRIS 0.015 M NaCl, pH 7.4) and 50 μl/well are added to the plate in triplicate. The human anti-K14 antibody (Lifespan Biosciences Inc, Seattle, Wash., US) is used as a positive control and is prepared in duplicate by serial dilution in sample buffer from 1:400 to 1:6400 in the plate. The plate is incubated at 37° C. for 2 hours. The washing steps are performed four times as described above.

The biotinylated detection antibody (Goat plyclonal to mouse IgG, IgM, IgA F(ab)2 fragment, Abcam, Cambridge, Mass., USA) is diluted 1/1500 in sample buffer and 50 μl is added to each well. The plate is incubated at 37° C. for 2 hours. The washing steps are performed four times as previously described.

Streptavidin-peroxidase is diluted 1:2000 in sample buffer and 50 μl is added to each well and the plate is incubated for 1 hour at RT. The washing steps are performed four times as described above.

A 1 mM ABTS solution is prepared in 70 mM citric buffer (pH 4.2) and protected from light (until use). 10 μl H₂O₂ (30%) is added to 10 ml of ABTS solution. Immediately after the addition of H₂O₂, 100 μl of the solution is added to each well in the plate. The plate is protected from light and incubated for 30 mM up to 1 hour. After that, the absorbance is measured at 405 nm in a plate reader (Spectramax, Molecular Devices Inc., Sunnyvale, Calif., USA).

The results are that the mice that have been exposed to haptens (on their skin) have antibodies against K14. This experiment shows that it is possible to map the epitopes also in human blood/sera.

### ELISA—Detection of Serum Amyloid Protein (SAP) in Sera

The SAP Mouse, ELISA kit 96 tests, quantitative (Alpha Diagnostics International Inc., San Antonio, Tex., USA) is used. The instructions provided with the kit are followed. Murine sera obtained in the experiment with booster dose on day 10 are used (see above).

The results are that exposing mice to oxazolone does not result in increased levels of SAP protein in the sera. Exposing mice to dBBr (a bidentate hapten) results in increased levels of SAP protein in sera.

### EXAMPLE 2

Diagnosis: Mapping Epitopes of Antibodies in Blood-Derived Sera from Hapten-Exposed Mice

### Epithelial Exposure of Various Compounds and Collection of Sera

Mice: CBA/Ca, females, 8-12 weeks old at the start of the experiment. (BK Scanbur, Sollentuna, Sweden).

Dissolve the chemical in molecular biology grade dimethyl sulfoxide (DMSO, Sigma Aldrich, Stockholm, Sweden) or other solvent recommended by OECD (e.g. acetone:olive oil 4:1) to yield the desired concentration.

For extreme sensitizers (EC3<0.1%): use a concentration 10 times the EC3-value. For strong sensitizers (0.1%<EC3<1%): use a concentration 10 times the EC3-value. For moderate sensitizers (1%<EC3<10%): use a concentration 2 times the EC3-value. For weak sensitizers (EC3>10%): use the same concentration as the EC3-value.

Examples of Compounds Used in Experiment with Booster Dose on Day 10:

Extreme sensitizers: Oxazolone (4-Ethoxymethylene-2-phenyl-2-oxazolin-5-one, 1.4 mM) CDNB (1-Chloro-2,4-dinitrobenzene, 7.4 mM)); Strong sensitizers: dBBr (48.6 mM), Glyoxal (ethane-1,2-dione, 1.27 M). DMSO is used as control. All chemicals are purchased from Sigma-Aldrich (Stockholm, Sweden).

### Procedure:

Apply 25 μl of the solution to the back of both ears (25 μl on each ear), once daily, on day 1, 2 and 3. Apply a booster dose on day 10 (if necessary). Sacrifice the mice on day 6 (or day 13 if a booster dose is used) by anesthetization with isoflurane (Kent Scientific Corporation, Torrington, Conn., USA) followed by inhalation of carbon dioxide (AGA, Lidingö, Sverige). To collect blood: Use a 1 ml syringe (B. Braun Medical Inc., Bethlehem, Pa., USA) with a 0.4 mm×12 mm injection needle (B. Braun Medical Inc., Bethlehem, Pa., USA). Place the tip of the needle just below the breastbone so that the syringe is linear to the abdomen. Insert the needle just below the breastbone and press it towards the heart. When inside the heart, withdraw blood. It is important to loosen the piston a little from the bottom of the syringe before inserting into the mouse. Carefully rotate the syringe during withdrawal to avoid clotting.

The blood is allowed to clot at room temperature for 30 minutes, then centrifuged at room temperature for 10 minutes at 2000 rpm using a Mikro 200R centrifuge (Hettich Zentrifugen, Tuttlingen, Germany). The serum is carefully pipetted off (using an automated pipette) and stored at −80° C. until use. Aliquot the samples to avoid excessive freeze-thaw treatment.

### Epitope Mapping:

Examples of proteins found in the blebs are keratins 5 and 14. The sequences of the keratins 5 and 14 are completely covered on the chip or plate using 8-mers (peptide sequences that are 8 amino acids long) with a stagger of three residues. Other proteins found in the blebs, are also sampled on the chip or plate. Other proteins (including, but not limited to homologues to the proteins found in the blebs, etc.) are also sampled on the chip or plate. In all cases, either relevant parts of the protein sequences are sampled, or the entirety of the protein in question is sampled (by overlapping peptide sequences).

For each peptide (i.e. epitope) the haptenated equivalent is also tested. Haptenation of the epitopes are done on the chip or plate by slowly running a solution of hapten dissolved in an appropriate solvent (for example dimethyl sulfoxide (DMSO, molecular biology grade, Sigma Aldrich, Stockholm, Sweden) and diluted in buffer (for example PBS, pH 7.4) over the microarray. The chip or plate is blocked with blocking buffer (SuperBlock (Thermo Scientific, Rockford, Ill.), 0.05% Tween-20, pH 7.0) overnight. Diluted blood-derived patient sample, e.g. serum (90 μg hemoglobin/ml) is then added to the chip or plate and incubated at room temperature for 1 hour. The chip or plate is then washed with washing buffer (PBS with 0.05% Triton X-100, 0.05% Tween-20, pH 7.0) at room temperature for 20 minutes. Incubate the chip or plate with 100 ng/ml secondary antibody (e.g. anti-mouse IgG+IGA+IgM Alexa 488 conjugate (Invitrogen, Carlsbad, Calif.)) in binding buffer (PBS pH 7.0) at room temperature for 20 minutes. Wash the chip or plate with washing buffer at room temperature for 20 minutes. The chip or plate is now ready for analysis which provides the basis for the diagnosis. In this case, fluorescence scanning detecting Alexa Fluor 488 is used. The fluorescence of the mouse serum sample is compared to controls. The amino acid containing epitope targets showing higher fluorescence than controls are regarded as positive (i.e. the mouse has antibodies against these epitopes). The result is that different sensitizing compounds give rise to different antibody binding patterns on the array and a diagnosis can thus be made.

### EXAMPLE 3

### Protocol for Induction of Tolerance in Mice

Mice: CBA/Ca, females, 8-12 weeks old at the start of the experiment. (BK Scanbur, Sollentuna, Sweden).

Peptides (including, but not limited to): 1: [Ace]-RISLGGACGAGGYG-[Amide]; 2: [Ace]-SLYNVGGSKRISYSS-[Amide], 3: [Ace]-DGKVVSTHEQVLRT-[Amide]. The peptides are custom synthesized and purified by ProImmune (Oxford, UK). The peptides 1 and 2 represent sequences from keratin 5 and peptide 3 is derived from keratin 14; however, it is the intention that the invention also includes peptides and/or proteins from other proteins, e.g. such as those found in the blebs from keratinocytes that have been exposed to haptens (sensitizing molecules).

### Modification of the Cysteine in Peptide 1:

Dissolve the peptide in pH 7.4 PBS (Sigma Aldrich, Stockholm, Sweden) to a concentration of 20 mM. Add mBBr (Sigma Aldrich, Stockholm, Sweden) to a concentration of 40 mM. Transfer the solution to a spin column with ˜10 μm pore size polyethylene filter (Pierce, ThermoFisher Scientific, Rockford, Ill., USA) containing immobilized TCEP disulfide reducing gel (Pierce, ThermoFisher Scientific, Rockford, Ill., USA) (1-2× the volume of peptide sample). Seal the tube and cover with aluminum foil and leave at room temperature for 2 hours with end-over-end mixing. Place the tube in a microfuge collection tube and centrifuge at 1000 rpm for 1 minute. Dialyze the solution against pH 7.4 PBS for 1 hour using a 500 Da molecular weight cut off membrane (“float-a-lyzer”, Spectrapor, Spectrum Labs, Rancho Dominguez, Calif., USA) to eliminate any unreacted or hydrolyzed mBBr. Lyophilize.

### Modification of Lysine in Peptide 2 and 3:

Same protocol as above but use an amine-reactive hapten, for example oxazolone (Sigma Aldrich, Stockholm, Sweden) instead.

### Modification of Arginine in Peptide 2 and 3:

Same protocol as above but use an arginine-reactive hapten, for example glyoxal (Sigma Aldrich, Stockholm, Sweden) instead.

### Tolerance

Divide the mice into two control groups and two test groups, each group consisting of 9 mice. Both control group receives phosphate-buffered saline (PBS) pH 7.4. The first control group is then topically exposed to DMSO. The second control group is then topically exposed to hapten of choice. One test group receives hapten-modified peptide and the other test group receives unmodified peptide. The test groups are then topically exposed to the hapten of choice. Dissolve the amino acid containing epitope targets in PBS pH 7.4 (Sigma Aldrich, Stockholm, Sweden) to yield a concentration of 0.2 mM to 100 mM. Tube-feed the mice with the peptide solutions once a day, for 5-20 consecutive days. Do not exceed 1 ml per 100 g of body weight. Apply hapten (dissolved in DMSO, Sigma Aldrich, Stockholm, Sweden) on the back of the ears on days 7-9. On day 12, inject 250 μl of an injection solution containing 80 μCi/ml [Methyl-³H]-thymidine (Perkin-Elmer, Waltham, Mass., USA) in sterile Dulbecco's PBS (Sigma-Aldrich, Stockholm, Sweden) into the tail vein. Let the mice rest for 5 hours. Sacrifice the mice by anesthetization with isoflurane (Kent Scientific Corporation, Torrington, Conn., USA) followed by inhalation of carbon dioxide (AGA, Lidingö, Sverige). Excise the local lymph nodes from each mouse and put into 1 ml of PBS, do not pool the nodes from different mice. Prepare single-cell suspensions of each pair of lymph nodes (i.e. each mouse) by pulping the nodes through a 70 μm cellstrainer (Becton Dickinson Labware, Franklin Lakes, N.J., USA) into a petri dish (NUNC, Sigma Aldrich, Stockholm, Sweden) using the backside of a piston from a 2 ml syringe (Codan, Kungsbacka, Sweden). Rinse the piston and cellstrainer with PBS (Sigma Aldrich, Stockholm, Sweden) and transfer the suspension to a round-bottomed centrifuge tube (10 ml, Sarstedt, Newton, N.C., USA). Add PBS to each tube to a total volume of 10 ml. Centrifuge for 10 min at 190×g and 4° C. Carefully discard the supernatant. Repeat this washing step twice. Remove the supernatant and add 3 ml of 5% (w/v) trichloroacetic acid (TCA) (Sigma Aldrich, Stockholm, Sweden). Shake up the pellet and let the tubes stand at 4° C. overnight. Centrifuge for 10 min at 190×g at 4° C. and remove as much supernatant as possible. Add 1 ml 5% TCA to each tube and transfer the pellet to 20 ml scintillation tubes (Zinsser Analytic, Frankfurt, Germany). Add 10 ml of Ecolume liquid scintillation cocktail (MP Biomedicals, Solon, Ohio, USA) and vortex thoroughly. Measure dpm in a scintillator and calculate the results according to OECD Guideline 429.

The results of the procedures are that the mice tube fed with amino acid containing epitope target show a lower amount of proliferated T cells compared to both control groups; hence sensitization is decreased due to oral tolerance.

### EXAMPLE 4

Diagnosis of ACD/Autoimmune Diseases/DHRs/etc. Using Human Blood-Derived Sera.

Blood samples from patients are collected and serum samples are prepared using standard protocols. Single serum specimens may not provide conclusive evidence and more samples may need to be collected and analyzed.

### Epitope Mapping:

Examples of proteins found in the blebs are keratins 5 and 14. The sequences of the keratins 5 and 14 are completely covered on the chip or plate using 8-mers (peptide sequences that are 8 amino acids long) with a stagger of three residues. Other proteins found in the blebs, are also sampled on the chip or plate. Other proteins (including, but not limited to homologues to the proteins found in the blebs, etc.) are also sampled on the chip or plate. In all cases, either relevant parts of the protein sequences are sampled, or the entirety of the protein in question is sampled (by overlapping peptide sequences).

For each peptide (i.e. epitope) the haptenated equivalent is also tested. Haptenation of the epitopes are done on the chip or plate by slowly running a solution of hapten dissolved in an appropriate solvent (for example dimethyl sulfoxide (DMSO, molecular biology grade, Sigma Aldrich, Stockholm, Sweden) and diluted in buffer (for example PBS, pH 7.4) over the microarray. The chip or plate is blocked with blocking buffer (SuperBlock (Thermo Scientific, Rockford, Ill.), 0.05% Tween-20, pH 7.0) over night. Diluted patient sample, e.g. serum (90 μg hemoglobin/ml) is the added to the chip or plate and incubated at room temperature for 1 hour. The chip or plate is then washed with washing buffer (PBS with 0.05% Triton X-100, 0.05% Tween-20, pH 7.0) at room temperature for 20 minutes. Incubate the chip or plate with secondary antibody (anti-human IgG Alexa 488, Invitrogen, Carlsbad, Calif., USA) in binding buffer (PBS pH 7.0) at room temperature for 20 minutes. Wash the chip or plate with washing buffer at room temperature for 20 minutes. The chip or plate is now ready for analysis which provides the basis for the diagnosis. In this case, fluorescence scanning detecting Alexa Fluor 488 is used. The fluorescence of the subject's biological sample is compared to controls. The amino acid containing epitope targets showing higher fluorescence than controls are regarded as positive (i.e. the patient has antibodies against these epitopes). The result is that allergies to different sensitizing compounds give rise to different antibody binding patterns on the array and a diagnosis can thus be made.

### EXAMPLE 5

### Diagnosis of ACD/Autoimmune Diseases/DHRs/etc Using Human Blood.

Blood samples from patients are collected and PBMCs are isolated and defibrinated using standard protocols. Single blood specimens may not provide conclusive evidence and more samples may need to be collected and analyzed.

T cells are added to a high throughput format plate (array) covered with amino acid containing epitope targets derived from K5, K14 or other protein released in blebs (with and without haptens). The cultures are incubated in RPMI#1640 medium with 2 mM glutamine, antibiotics, 5 mM HEPES buffer and 10% human serum for four to six days at 37° C. in a humidified 5% CO₂ incubator. The T cells are then labeled with 0.25 mCu of tritiated thymidine (>40 Ci/mmol) for 6 h before harvesting the DNA according to standard procedures and scintillation counting. The stimulation result is compared to control experiments consisting of unstimulated T cell cultures. The result is that allergies to different sensitizing compounds give rise to different T cell binding patterns on the array and a diagnosis can thus be made.

### EXAMPLE 6

### Protocol for Induction of Oral Tolerance in Humans

Peptides (including, but not limited to): 1: [Ace]-VSLGGACGAGGYG-[Amide]; 2: [Ace]-SLYNLGGSKRISIST-[Amide], 3: [Ace]-DGKVVSTHEQVLRT-[Amide]. The peptides 1 and 2 represent sequences from keratin 5 and peptide 3 is derived from keratin 14; however, it is the intention that the invention also includes peptides and/or proteins from other proteins, e.g. such as those found in the blebs from keratinocytes that have been exposed to haptens.

Preparation of amino acid containing epitope targets as described in example 3.

### Tolerance

Of 20 individuals with a confirmed allergy to the hapten cinnamic aldehyde, 10 are given a test solution of 0.5 mg to 10 g amino acid containing epitope target 1 once a day for 5-100 days. The other 10 individuals are given a placebo (i.e. tap water) to drink according to the same administration scheme and time table as described above. The antibody and T cell epitope mapping profiles for each individual are then evaluated according to procedures in example 4 and 5. Each individual is also patch-tested with cinnamic aldehyde. The results are that the individuals who have been drinking test solution show less antibodies and T cells against the amino acid containing epitope target and less reaction to cinnamic aldehyde in the patch test.

What is claimed is:

1. A method of determining a patient's profile in vitro, comprising:
a) providing a high-throughput format containing an array of different amino acid-containing epitope targets released in blebs and selected for testing for the presence of particular antibodies in the patient, wherein the amino acid-containing epitope targets are positioned on the format so that the exact position of each epitope on the format is known;
b) adding a blood-derived sample from the patient to the high-throughput format containing the selected epitope targets under conditions conducive to binding of the epitope targets to antibodies or T-cells;
c) analyzing the high-throughput format for antibodies or T-cells that bind to the epitope targets by detecting whether there is a detectable signal due to the binding of the epitope targets by antibodies or T-cells;
d) comparing signals obtained from standard known samples with signal detected from the patient blood-derived sample to determine which antibodies or T-cells are in the patient blood-derived sample; and
e) using the determination of which antibodies are in the patient blood-derived sample to determine to which epitope targets the patient is allergic.

2. The method of claim 1, further comprising using the determination of which antibodies or T-cells are in the blood-derived sample to diagnose the allergen(s) in a condition selected from the group consisting of allergic contact dermatitis, drug hypersensitivity reaction, and autoimmune diseases.

3. The method of claim 1, wherein the high-throughput format is a microfluidic or nanofluidic chip.

4. The method of claim 1, wherein the high-throughput format is a microtiter plate.

5. The method of claim 1, wherein the epitope is a haptenated epitope.

6. The method of claim 1, wherein the epitopes are selected from the group consisting of keratin 5, keratin 14, keratin 1, keratin 10, keratin 8, keratin 18, glucose-6-phosphatase isomerase, transitional endoplasmic reticulum ATPase, protein disulfide isomerase, calmodulin, importin 5, keratin 6 (A, B and C), stathmin, transgelin-2, 14-3-3 protein sigma, calreticulin, endoplasmin, heat shock protein 90, actin beta, actin gamma, alpha actinin, cofilin 1, ezrin, fibronectin, myosin 1c, plastin 2, plastin 3, tubulin beta 2C, annexin 2, peroxiredoxin 1, SSA/Ro ribonucleoprotein, alpha enolase, peptidyl-prolyl cis-trans isomerase A.

7. The method of claim 1, wherein the amino acid-containing sequences are obtained by a method selected from the group consisting of recombination and direct purification from cells or animals.

8. The method of claim 1, wherein the analyzing for antibodies or T-cells comprises using detection antibodies that bind the antibodies in the blood-derived sample to produce a detectable signal.

9. The method of claim 8, wherein the detection antibodies carry a detection substance selected from the group consisting of fluorescent probes, biotin and enzyme substrates.

10. The method of claim 8, further comprising using a secondary binder that binds to the detection antibody and that is linked to an enzyme that is detectable using an enzyme substrate that forms chromogenic end products, and adding the enzyme substrate.

11. A kit for determining a patient's antibody or T-cells profile, comprising:
a) a high-throughput format containing an array of different amino acid-containing epitope targets released in blebs and selected for testing for the presence of particular antibodies or T-cells in the patient, wherein the amino acid-containing epitope targets are positioned on the format so that the exact position of each epitope on the format is known;
b) a selection of detection antibodies that bind the antibodies or T-cells in the blood-derived sample to produce a detectable signal; and
c) blocking, binding and washing buffers.

12. The kit of claim 11, wherein the high-throughput format is a microfluidic or nanofluidic chip.

13. The kit of claim 11, wherein the high-throughput format is a microtiter plate.

14. The kit of claim 11, wherein the epitope is a haptenated epitope.

15. The kit of claim 11, wherein the high-throughput format is coated with bleb proteins.

16. The kit of claim 11, wherein the high-throughput format is coated with K5 and K14 peptides, and/or haptenated K5 and K14 peptides.

17. The kit of claim 11, wherein the detection antibodies carry a detection substance selected from the group consisting of fluorescent probes, biotin, and enzyme substrates.

18. The kit of claim 11, further comprising: i) a secondary binder that binds to the detection antibody and that is linked to an enzyme that is detectable using an enzyme substrate that forms chromogenic endproducts, and ii) the enzyme substrate.

19. The kit of claim 11, wherein at least one of the amino acid-containing epitope targets is a haptenated amino acid-containing epitope target.

20. A method for identifying targets to induce tolerance, comprising determining a patient's profile according to claim 1 to determine to which epitope targets the patient is allergic, and orally administering to the patient an epitope target to which the patient is allergic in sufficient amounts to induce tolerance to the epitope target

21. The method according to claim 20 where tolerance is induced against a condition selected from the group consisting of allergic contact dermatitis, drug hypersensitivity reaction, and autoimmune diseases.
